(19) United States
(12) Patent Application Publication
(10) Pub. No.: US 2026/0232908 A1
(43) Pub. Date: Aug. 13, 2026

(54) SYRINGE DEVICE FOR DISPENSING FLUIDS

(71) Applicant: AMPULIS INC., Berwyn, PA (US)

(72) Inventor: Eric SUGALSKI, Berwyn, PA (US)

(21) Appl. No.: 19/573,141

(22) Filed: Mar. 20, 2026

Related U.S. Application Data
(63) Continuation of application No. PCT/US2025/024024, filed on Apr. 10, 2025.
(60) Provisional application No. 63/696,990, filed on Sep. 20, 2024.

(51) Int. Cl. A61M 5/24; A61J 1/06; A61M 5/19; A61M 5/31; A61M 5/315

(52) U.S. Cl. CPC A61M 5/2448; A61J 1/065; A61M 5/19; A61M 5/2422; A61M 5/3134; A61M 5/3145; A61M 5/31511; A61M 2005/2407; A61M 2005/2474; A61M 2005/3115; A61M 2005/3128; A61M 2202/064; A61M 2205/586; A61M 2205/7545

(57) ABSTRACT

A compact syringe device for dispensing fluids in a precise manner includes a syringe body having a barrel body defining a dispensing chamber and a storage body defining a dispensing chamber, with a common wall of the syringe body extending from the proximal ends to the distal ends of the chambers to separate and define them. The syringe device may include a check valve resisting backflow into the storage chamber, a plunger slidable within the barrel body, and a cap sealing the storage chamber. A breaker lever may be provided to apply force to the storage chamber to break a frangible ampoule positioned therein. Alternatively, a rotatable ampoule carrier may be provided in the storage body that includes resilient fingers displaceable inwardly during rotation to crush an ampoule. A spring-biased mechanism may be provided for moving the plunger to draw liquid from a fractured ampoule into the dispensing chamber.

### CROSS-REFERENCE TO RELATED APPLICATION

The application is a continuation of International Application No. PCT/US2025/024024, filed on Apr. 10, 2025, and claims the benefit of priority, under 35 U.S.C. § 119(e), of U.S. Provisional Patent Application No. 63/696,990, filed Sep. 20, 2024, each of which are incorporated herein by reference in their entirety.

### FIELD OF THE INVENTION

The present invention relates generally to fluid delivery devices, and more particularly to a syringe-type injection device for dispensing drugs, biologics or other fluids, at least a part of which may be packaged in ampoules that can be crushed.

### DISCUSSION OF RELATED ART

Syringe devices have many uses for dispensing fluids. Certain uses include dispensing fluids that are packaged. In certain cases, the dispensed fluid may be result of mixing one or more fluids and/or dry powders. By way of example, the syringe devices may be used to dispense fluids onto a surface, e.g., topically, and in the case of medicinal products, may be used to inject fluids into an intravenous line or otherwise via a luer-type connector, or into bodily tissue (e.g., subcutaneously, intramuscularly and/or transdermally), e.g., via a needle of the syringe device.

Ampoules are often used to package and store materials/products in liquid form in relatively small quantities (e.g., dose volumes), e.g., about 0.1 to about 2.0 ml. It should be noted, however, that ampoules may also be used for the storage of powders, and therefore, the terms liquid and powder herein may often be used interchangeably. Ampoules are often used, for example, to store chemical or medicinal products.

Ampoules are typically made of glass or plastic and define an internal cavity that is hermetically sealed from the environment by the ampoule material. A benefit of ampoules is they provide tamper resistant packaging and the ability to immediately detect when the package has been compromised. Ampoules are also used to protect sensitive formulations from environmental exposure. Furthermore, the singular glass and plastic material composition reduces the risk of chemical interactions, such as leachables, that could degrade the contained material.

The ampoule is formed as a containment vessel for the liquid and is initially fabricated with one end open for liquid filling. At the time the ampoule is filled with liquid, the open end is heat-formed to hermetically seal its contents. The liquid then remains stored in the ampoule until it is used for its intended purpose. Generally, the ampoule is subsequently ruptured by manually breaking the ampoule body immediately before using the liquid, so that the internal cavity portion of the ampoule becomes accessible such that the liquid contained therein can flow out for use.

Various devices, many including filters, are used with crushable ampoules to protect the user from glass fragments while still permitting a liquid or gel (e.g., therapeutic) to be used for either application or creation of a vapor, such as smelling salts.

Liquids stored in ampoules may be used in many different manners, and for many different purposes. In certain applications, it is desirable to be able to combine one or more liquids with one or more liquids and dry powders to form a fluid, and to dispense the fluid in a precisely-controlled manner, e.g., to provide a controlled-dose injection. An example of such an application is injection of a drug-containing fluid to an intended area of a body. It should be noted, however, that biologic-containing fluids follow a similar injection process, and therefore, the terms drugs and biologics herein may often be used interchangeably. In such circumstances, it is desirable to dispense the drug from a syringe in a precisely-controlled manner so that dosing of either too little, or too much, drug to a person's epidermis, dermis, subcutaneous, muscle, or other body location can be avoided. In other words, in certain applications, it is desirable to be able to dispense a drug in fluid form in a manner from a syringe to ensure drug dose accuracy.

Many drugs with stability challenges are stored in liquid and powder forms within vials. For these drugs, a user must source accessory syringes and needle sets to access the drugs within these vials. Furthermore, many drugs stored in a powder (or lyophilized) form require additional steps to introduce diluent to convert the lyophilized drug into a liquid (or reconstituted) form. These processes involving accessing drug from vials and reconstituting drugs introduce risks of contamination and potential error which can affect the dose accuracy and adherence of healthcare providers and patients performing self-administration.

What is needed is a compact device for storing, mixing and/or dispensing fluids, and one that may be used to dispense liquids or powders contained in crushable ampoules that can be used to rupture the ampoule to release the liquid/powder, to filter glass particles from the ruptured ampoule, and to dispense a fluid from a syringe device in a precisely-controlled manner to help ensure fluid (e.g., drug) delivery accuracy.

### SUMMARY

The present invention provides a compact syringe-type device configured for dispensing fluids in a precisely-controlled manner to help ensure fluid delivery accuracy, particularly when the fluid is a drug, to ensure dose accuracy. Further, the present invention provides a syringe device that may be used to dispense fluids packaged in crushable ampoules, and more particularly, a syringe device that can be used to rupture the ampoule to release a liquid or dry powder, to filter glass particles from the ruptured ampoule, and to dispense a fluid from the syringe device.

In certain embodiments, the syringe device may be prefilled with one or more liquids and/or dry powders, to store and serve as packaging for such materials. In other embodiments, the syringe device may be configured to receive liquids and/or dry powders packaged in frangible ampoules. In either case, the syringe device may be used in a manner that causes a liquid and powder, or multiple liquids, to be mixed within a dispensing chamber of the syringe device before the resulting combination is dispensed as a fluid from the syringe device.

A syringe body in accordance with the present invention includes: a barrel body defining a dispensing chamber open at a first proximal end and having a first distal end terminating in a dispensing tip defining a first flow passage, the dispensing chamber being in fluid communication with the first flow passage, the dispensing chamber being elongated to have a first longitudinal axis; and a storage body defining a storage chamber having a second proximal end and a second distal end, the storage chamber defining an opening at one of the second proximal end and the second distal end, the storage chamber being elongated to have a second longitudinal axis distinct from the first longitudinal axis; wherein a common wall of the syringe body extends from the first and second proximal ends to the first and second distal ends to separate and at least partially define both the dispensing chamber and the storage chamber, and wherein a second flow passage is defined by the syringe body to provide fluid communication between the storage chamber and the dispensing chamber.

A syringe device in accordance with the present invention may include not only a syringe body but also: a flow check valve supported on the syringe body, the flow check valve being positioned to resist backflow of liquid/fluid into the storage chamber; a plunger positioned within the barrel body for longitudinal sliding movement therein, the plunger and extending beyond the first proximal end of the barrel body, the plunger comprising: a plunger rod having a third proximal end and a third distal end; and a plunger tip supported on the third distal end of the plunger rod, the plunger tip being configured to abut and seal with an internal sidewall of the barrel body; and a cap disposed within the opening at a respective one of the second proximal end and the second distal end of the storage body to seal the storage chamber.

In certain embodiments, the syringe device includes a breaker lever mounted to the syringe body, the breaker lever defining a protrusion positioned and configured to apply force to the flexible portion of the outer wall to displace the outer wall inwardly and break a frangible ampoule positioned within the storage chamber when the breaker lever is adducted toward the storage chamber. The breaker lever is operable between at a stored position and an ampoule break position.

In other embodiments, the syringe device includes a rotatable ampoule carrier that includes resilient fingers that are displaceable inwardly during rotation to crush a frangible ampoule contained within the ampoule carrier.

In still other embodiments, the syringe device includes a spring-biased mechanism for automatedly moving the plunger to draw a vacuum that draws liquid from a fractured ampoule in the storage chamber into the dispensing chamber.

### BRIEF DESCRIPTION OF THE FIGURES

An understanding of the following description will be facilitated by reference to the attached drawings, in which:

FIG. 1 is a perspective cross-sectional view of a syringe device in accordance with an exemplary embodiment of the present invention, showing the syringe device in cross-section;

FIG. 2 is an exploded cross-sectional view of the syringe device of FIG. 1;

FIG. 3 is a side cross-sectional view of the syringe device of FIG. 1, showing how a frangible ampoule may be loaded into a storage chamber of the syringe device;

FIG. 4A is a side cross-sectional view of the syringe device of FIG. 1, showing a dry powder loaded into a dispensing chamber of the syringe device for mixing with a liquid stored in a frangible ampoule loaded into a storage chamber of the syringe device;

FIG. 4B is a side cross-sectional view of the syringe device of FIG. 1, showing the dry powder and liquid of FIG. 4A mixed in the dispensing chamber of the syringe device;

FIG. 5 is a side cross-sectional view of an alternative syringe device in accordance with an alternative exemplary embodiment of the present invention, showing an integral breaker lever joined by a living hinge, a dispensing tip, and a luer connector;

FIG. 6 is a side view of another alternative syringe device in accordance with another alternative exemplary embodiment of the present invention, showing a separate breaker lever joined to a syringe body of the syringe device;

FIG. 7 is a side cross-sectional view of another alternative embodiment of a syringe device, showing a first liquid loaded into a storage chamber of the syringe device;

FIG. 8 is a side cross-sectional view of the syringe device of FIG. 7, showing a second liquid loaded into a dispensing chamber of the syringe device for mixing with a first liquid stored in a storage chamber of the syringe device;

FIG. 9 is a side cross-sectional view of the syringe device of FIG. 1, showing a dry powder loaded into a dispensing chamber of the syringe device for mixing with a first liquid loaded into a storage chamber of the syringe device;

FIG. 10 is a side cross-sectional view of another alternative embodiment of a syringe device, showing a first liquid loaded into a storage chamber of the syringe device, and a floating plunger tip in the storage chamber;

FIGS. 11 and 12 are side cross-sectional views of the syringe device of FIG. 10, showing the first liquid from the storage chamber being drawn into the dispensing chamber of the syringe device;

FIGS. 13 and 14 are side and top cross-sectional views, respectively, of another alternative syringe device in accordance with another alternative exemplary embodiment of the present invention, showing a storage chamber configured to hold multiple ampoules and a separate breaker lever joined to a syringe body of the syringe device;

FIG. 15 is a side cross-sectional view of another alternative embodiment of a syringe device, showing a first liquid loaded into a storage chamber of the syringe device, a dry powder loaded into a dispensing chamber of the syringe device, and a respective plunger in each of the storage and dispensing chambers;

FIG. 16 is a side cross-sectional view of the syringe device of FIG. 15, showing a first liquid loaded into a storage chamber of the syringe device, showing the dry powder and liquid of FIG. 15 mixed in the dispensing chamber of the syringe device;

FIG. 17 is a side cross-sectional view of another alternative embodiment of a syringe device for integration with an autoinjector device, showing a first liquid loaded into a storage chamber of the syringe device, a dry powder loaded into a dispensing chamber of the syringe device, and a respective floating plunger tip in each of the storage and dispensing chambers;

FIG. 18 is an exploded perspective view of another alternative syringe device in accordance with another alternative exemplary embodiment of the present invention, showing an ampoule carrier including resilient fingers operable to crush a frangible ampoule contained therein;

FIGS. 19-21 are perspective, top and side views of the syringe device of FIG. 18, showing the syringe device in a storage state;

FIG. 22 is a cross-sectional view taken along line 22-22 of FIG. 21;

FIG. 23 is an enlarged cross-sectional view of portion of FIG. 22;

FIGS. 24-26 are perspective, top and side views of the syringe device of FIG. 18, showing the syringe device in a crushed ampoule state;

FIG. 27 is a cross-sectional view taken along line 27-27 of FIG. 26;

FIG. 28 is an enlarged cross-sectional view of portion of FIG. 27;

FIG. 29 is an exploded view of a syringe device in accordance with yet another exemplary embodiment of the present invention;

FIG. 30 is a front cross-sectional view of the syringe device of FIG. 29, taken along line 30-30 of FIG. 29, showing the syringe device and ampoule in a stored state;

FIG 31. is a cross-sectional view of the syringe device of FIG. 29, taken along line 31-31 of FIG. 30;

FIG. 32 is an enlarged view of a portion of FIG. 31;

FIG. 33 is a front cross-sectional view of the syringe device, as shown in FIG. 30;

FIG. 34 is an enlarged view showing a lower portion of the syringe device of FIG. 33;

FIG. 35 is an enlarged view showing an upper portion of the syringe device of FIG. 33;

FIG. 36 is a front cross-sectional view of the syringe device of FIG. 29, showing the syringe device in a cracking state and the ampoule in a ruptured state;

FIG. 37 is a cross-sectional view of the syringe device taken along line 37-37 of FIG. 36;

FIG. 38 is an enlarged view of a portion of FIG. 37;

FIG. 39 is a front cross-sectional view of the syringe device of FIG. 29, showing the syringe device in a vacuum state and the ampoule in ruptured state;

FIG. 40 is a cross-sectional view of the syringe device taken along line 40-40 of FIG. 39;

FIG. 41 is an enlarged view of a portion of FIG. 40;

FIG. 42 is a front cross-sectional view of the syringe device of FIG. 29, showing the syringe device in a dispense state and the ampoule in an empty state;

FIG. 43 is a cross-sectional view of the syringe device of FIG. 29, taken along line 43-43 of FIG. 42; and

FIG. 44 is an enlarged view of a portion of FIG. 43.

### DETAILED DESCRIPTION

The present invention provides a compact syringe-type device configured for dispensing fluids in a precisely-controlled manner to help reduce contamination and ensure fluid delivery accuracy, particularly when the fluid is a drug, to ensure safety and dose accuracy. Further, the present invention provides a syringe device that may be used to dispense liquids or dry powders packaged in crushable ampoules, and more particularly, that can be used to rupture the ampoule to release the liquid/powder, to filter glass particles from the ruptured ampoule, and to dispense a fluid from the syringe device.

In certain embodiments, the syringe device may be prefilled with one or more liquids and/or dry powders, to store and serve as packaging for such materials. In other embodiments, the syringe device may be configured to receive liquids and/or dry powders packaged in frangible ampoules. In either case, the syringe device may be used in a manner that causes a liquid and powder, or multiple liquids, to be mixed within the syringe device before the resulting mixed fluid is dispensed from the syringe device.

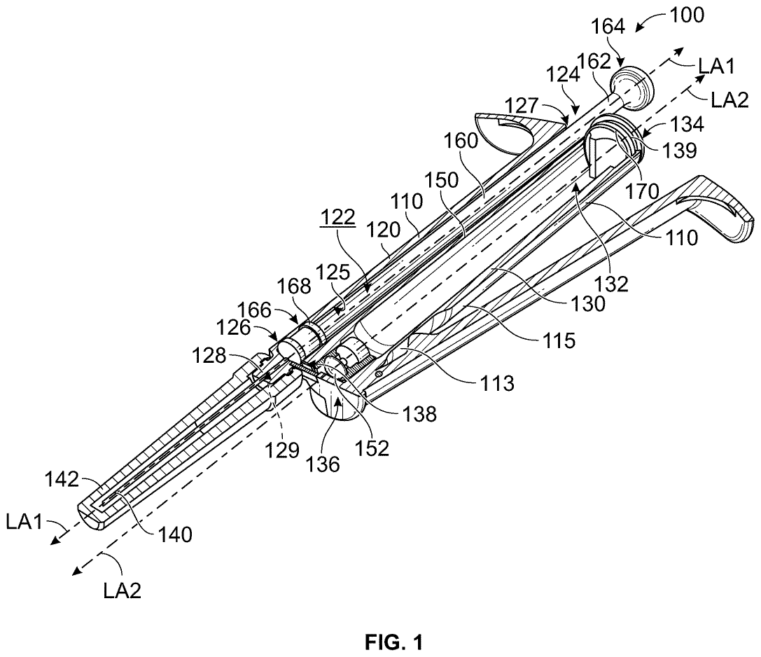

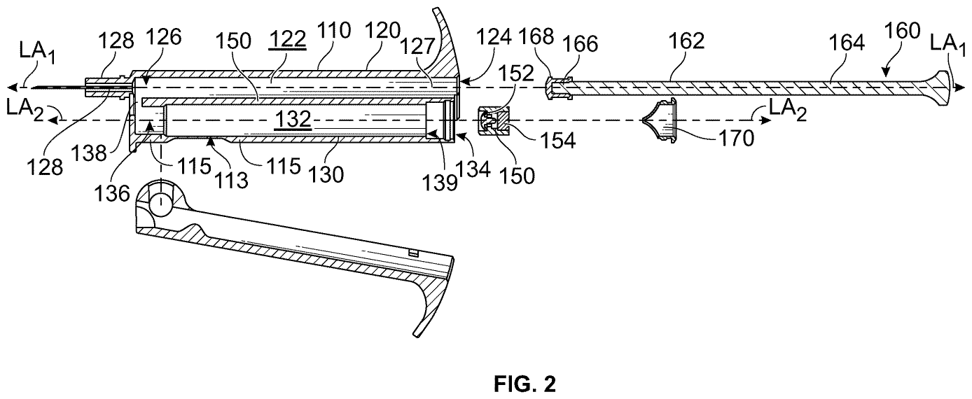

FIGS. 1-3 show an exemplary embodiment of a syringe device 100 in accordance with an exemplary embodiment of the present invention. As will be appreciated from FIGS. 1-3, the syringe device includes a syringe body 110.

The syringe body comprises a barrel body 120. The barrel body 120 defines a dispensing chamber 122 open at a first proximal end 124 and having a first distal end 126. The barrel body 120 defines an opening 127 at the first proximal end 124, and terminates in a dispensing tip 128 defining a first flow passage 129. The dispensing chamber 122 is in fluid communication with the first flow passage 129. As will be appreciated from FIG. 1, the dispensing chamber 122 is elongated along a first longitudinal axis LA1.

In this exemplary embodiment, the syringe device 100 further includes a plunger 160. The plunger 160 includes a plunger rod 162 having a respective proximal end 164 and a respective distal end 166. A plunger tip 168 is supported on the distal end 166 of the plunger rod 162. The plunger tip 168 is configured to abut and seal with an internal sidewall 125 of the barrel body 120. By way of example, the plunger tip 168 may be formed of an elastomeric material. The internal sidewall 125 defines the dispensing chamber 122, which may or may not be circular in cross-section. As will be appreciated from FIGS. 1-3, the plunger 160 is positioned within the barrel body 120 for longitudinal sliding movement therein, with the plunger 160 extending through the opening 127, and beyond the first proximal end 124, of the barrel body 120.

In this exemplary embodiment, the syringe device includes a needle 140, e.g., a needle usable for subcutaneous, intramuscular or transdermal injection of a drug into bodily tissue. The needle 140 is supported on the dispensing tip 128 (e.g., inserted with a friction fit, adhered in-place by an adhesive, or inserted into the injection mold tool prior to injection molding), such that the first flow passage 129 is defined at least in part by a lumen of the needle 140, such that the first flow passages 129 passes through the needle 140. In this case, a needle cover/shield 142 is releasably supported on the dispensing tip 128 with a snap/interference fit, such that it can be easily removed manually prior to delivery of an injection, in a manner generally known in the art.

Further, the syringe body 110 defines a storage body 130. The storage body 130 defines a storage chamber 132. The storage chamber 132 has a respective (second) proximal end 134 and a respective (second) distal end 136. The storage body 130 defines an opening 139 at one of the second proximal end 134 and the second distal end 136. In this embodiment, the opening 139 is defined at the second proximal end 134, as will be appreciated from FIG. 1. The storage chamber 130 is elongated along a second longitudinal axis LA2 distinct from the first longitudinal axis LA1. In this case, axes LA1 and LA2 are defined in a spaced parallel relationship, although in other embodiments, they may not be so defined.

In accordance with the present invention, a common wall 150 of the syringe body 110 extends from the first and second proximal ends 124, 134 to the first and second distal ends 126, 136 to separate and at least partially define both the dispensing chamber 122 and the storage chamber 132. In certain embodiments, the common wall 150 may be elongated laterally to form a bridge between the barrel body 120 and storage body 130 that increases a distance between longitudinal axes LA1 and LA2 beyond a distance between them when the barrel body and storage body are abutting one another, so that the bridge acts as a lateral spacer of the axes LA1 and LA2 and the barrel and storage bodies 120, 130.

A second flow passage 138 is defined by the syringe body 110 to provide fluid communication between the storage chamber 132 and the dispensing chamber 122. This allows liquid in the storage chamber to be drawn into the dispensing chamber 122 for mixing and/or dispensing purposes, as described further herein.

In the exemplary embodiment of FIGS. 1-3, a flow check valve 152 is supported on the syringe body 110. The flow check valve 152 is operatively positioned in the adjacent the second flow passage 138 to resist backflow of liquid/fluid into the storage chamber 122. In the exemplary embodiment of FIGS. 1-3, the flow check valve 152 is positioned, by way of example, within the storage chamber 132. Further, the flow check valve 152 is provided as an umbrella-type valve by way of example. It will be appreciated by those skilled in the art that other types of flow check valves may be employed, and that the flow check valve may be positioned other than in the storage chamber 132, in accordance with the present invention.

The syringe device 100 further includes a cap 170 disposed within the opening 139 of the storage body 130, which in this example is at the distal end 134 of the storage body 130. The cap 170 seals the storage chamber 132 against the atmosphere/ambient air, e.g., to prevent dust/dirt/debris, etc. from entering the storage chamber 132 and contaminating its contents. The cap 170 may have various forms, as discussed in greater detail below.

In this exemplary embodiment, the syringe device 100 is adapted for use in conjunction with a frangible ampoule 10 that contains a liquid L1, such that the ampoule serves as the primary liquid container, and such that the syringe device 100 can be used to dispense the liquid L1 stored in the ampoule 10, e.g., as a drug delivery device. Various conventional, commercially-available frangible/crushable ampoules are known in the art, and are beyond the scope of the present invention, and thus are not discussed in detail herein.

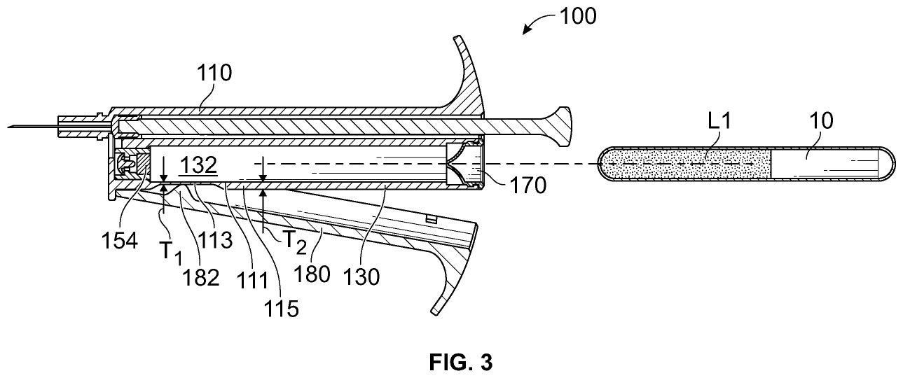

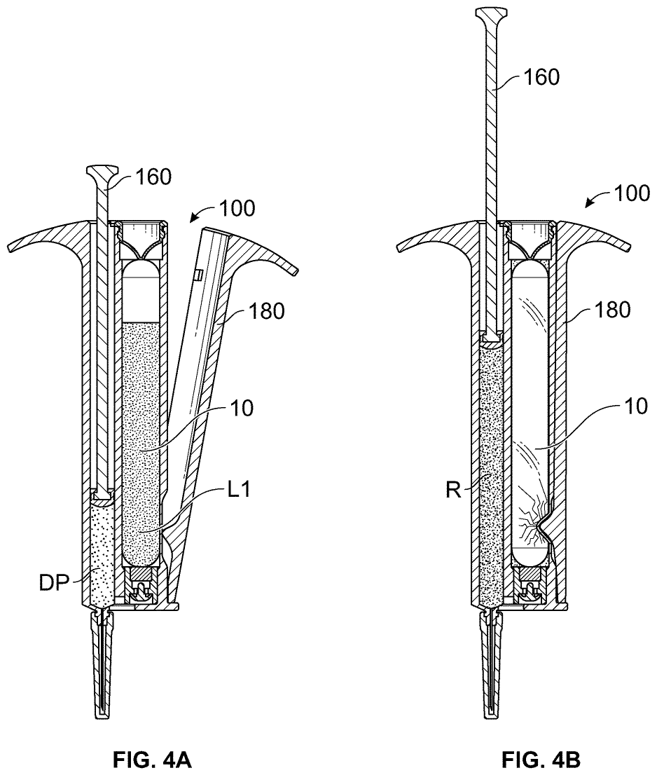

FIG. 3 is a sectional view of the syringe device 100 of FIG. 1, showing how a frangible ampoule 10 may be loaded into the storage chamber 132 of the syringe device 100. Accordingly, in this exemplary embodiment, the cap 170 is provided as an entry port that is configured to open to admit passage of a frangible ampoule into the storage chamber 132 through the entry port, and to close to seal the storage chamber 132 when an ampoule is not positioned within the entry port. Although the entry port may have any suitable configuration in accordance with the present invention, in the exemplary embodiment of FIGS. 1-3, the cap/entry port 170 is constructed of an elastomeric material and configured as a duckbill valve. Accordingly, in this embodiment, a frangible ampoule 10 may be loaded into the storage chamber 130 of the syringe device 100 by pushing the ampule 10 through the cap/entry port 170 into the storage chamber 132 until the ampoule 10 clears the duckbill valve structure, at which point the duckbill valve/entry port closes to seal the storage chamber 130 with the ampoule 10 contained therein, as shown in FIG. 4A.

Because this embodiment is intended to be used with a frangible ampoule that is intended to be crushed and ruptured to release the liquid L1 contained therein, this exemplary embodiment of FIGS. 1-3 further includes a particulate filter 154 that is also supported on the syringe body 110. The particulate filter 154 is operatively positioned adjacent to the second flow passage 138 to prevent any broken glass or other particles from any ruptured ampule in the storage chamber 132 from exiting the syringe device 100 via the dispensing tip 128. In the exemplary embodiment of FIGS. 1-3, the particulate filter 154 is positioned, by way of example, within the storage chamber 132. Further, the particulate filter 154 is provided as a disc of sintered polytetrafluoroethylene (PTFE) by way of example. It will be appreciated by those skilled in the art that other types of particulate filters may be employed, and that the particulate filter may be positioned other than in the storage chamber 132, such as within the first flow passage 129 (as best shown in FIG. 1), in accordance with the present invention.

In this exemplary embodiment, the syringe device 100 is further adapted for use with a frangible ampoule in that the syringe body 110 includes an outer wall 111 that at least partially defines the storage chamber 132 and that defines a flexible portion 113 that is displaceable inwardly (into the storage chamber 132) to apply pressure to a frangible ampoule 10 positioned within the storage chamber 130 to cause it to rupture and release its contents. In the example of FIGS. 1-3, the flexible portion 113 of the outer wall 111 has a thickness T1 that is reduced relative to a respective thickness T2 of an adjacent portion 115 the outer wall 111, such that the reduced thickness portion T1 is relatively more flexible than the greater thickness portion T2, as will be appreciated from FIG. 3.

In this exemplary embodiment, the syringe device 100 is further adapted for use with a frangible ampoule in that the syringe device 100 further includes a breaker lever 180 mounted to the syringe body 110. The breaker lever 180 defines a protrusion 182 positioned and configured to apply force to the flexible portion 113 of the outer wall 111 to displace the flexible portion 113 of the outer wall 111 inwardly and break a frangible ampoule 10 positioned within the storage chamber 132 when the breaker lever 180 is adducted toward the storage chamber 132. Accordingly, the breaker lever 180 is operable between a stored position (e.g., as shown in FIGS. 1-4A) and an ampoule break position (as shown in FIG. 4B).

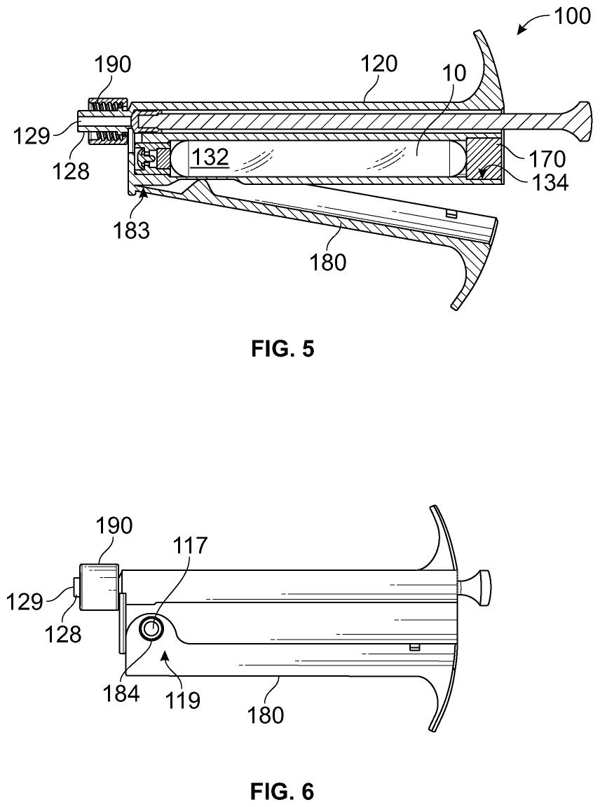

In certain embodiments, the breaker lever 180 is integrally formed with, and an integral part of, the syringe body 110, as shown in FIG. 5. In such an embodiment, the breaker lever 180 may be constructed of a relatively flexible material or may otherwise be connected via a thinned portion such that the breaker level 180 is connected to a remainder of the syringe body 110 by, and therefore pivotable about, a living hinge 183 formed by the thinned portion, as best shown in FIG. 5.

In other embodiments, the breaker lever 180 is a discrete component separate from the syringe body 110, as shown in FIG. 6. In such an embodiment, the breaker lever 180 may be constructed of a rigid material and is pivotably interconnected to the syringe body 110 by a pivot joint 119, which may be formed, by example, by a complementary pivot pin and socket, such as a pivot pin 117 on the syringe body and a complementary socket/opening 184 defined on the breaker level 180, as best shown in FIG. 6.

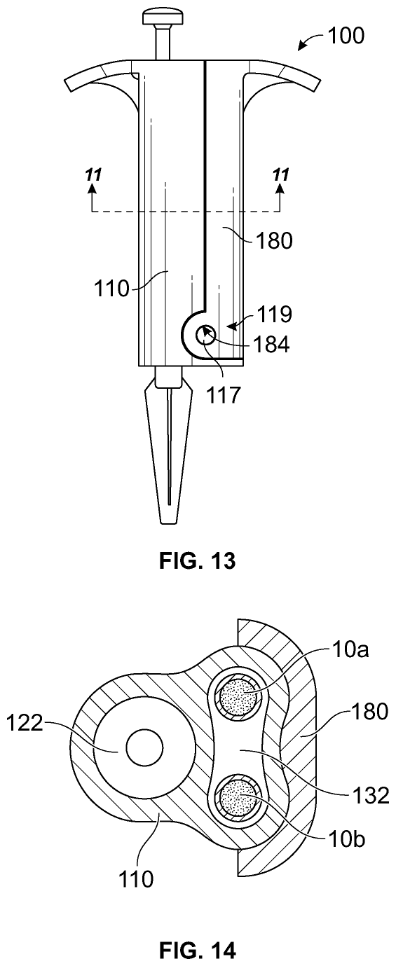

In certain embodiments, the storage body 130 and storage chamber 132 are dimensioned to receive at least two frangible ampoules. FIGS. 13 and 14 are side and top views of an alternative syringe device 100 including a storage chamber 132 configured to hold multiple ampoules. As will be appreciated from FIGS. 13 and 14, in this embodiment, the breaker lever 180 (in this example, a separate discrete breaker level 180) is joined to a syringe body of the syringe device and is configured such that it will apply force to the flexible portion 113 of the outer wall 111 to displace the outer wall inwardly and break both frangible ampoules 10a, 10b positioned within the storage chamber 132 when the breaker lever 180 is adducted toward the storage chamber 130. This will cause the contents of the ruptured frangible ampoules to be released concurrently to cause them to mix, which may be desirable in certain embodiments.

As referenced above, the syringe device may be prefilled with one or more liquids and/or dry powders, and/or may be configured to receive liquids and/or dry powders packaged, to store and serve as packaging for such materials, and/or to otherwise causes a liquid and a dry powder, or multiple liquids, to be mixed within the dispensing chamber 122 of the syringe device 100 before a resulting mixed fluid is dispensed from the syringe device 100.

FIGS. 1-3 illustrate an exemplary use of the syringe device to receive a liquid L1 packaged in a frangible ampoule 10, to rupture the ampoule 10, and to dispense the packaged liquid L1. By way of further example, FIG. 4a is a sectional view of the syringe device of FIGS. 1-3, but showing a dry powder DP may be disposed in the dispensing chamber 122 of the syringe device 100 for mixing with a first liquid L1 stored in a frangible ampoule 10 loaded into a storage chamber 132 of the syringe device. For example, the dry powder may be a lyophilized drug, and the first liquid may be a diluent, such that operation of the breaker level 180 to rupture the ampoule 10 and release the diluent into the storage chamber 132, and operation of the plunger 160 to draw the diluent from the storage chamber 132 into the dispensing chamber 122 causes the lyophilized drug and the diluent to be mixed in the dispensing chamber 122 to form a reconstituted drug R that is a fluid dispensable by the syringe device 100, as will be appreciated from FIGS. 4A and 4B. By way of alternative example, a second liquid L2 may be loaded into the dispensing chamber 122 of the syringe device 100 instead of the dry powder for mixing with a first liquid L1 stored in a frangible ampoule 10 loaded into a storage chamber 132 of the syringe device, such that the resulting mixed fluid may be dispensable via the syringe device 100.

FIGS. 5 and 6 illustrate an alternative syringe device 100 that includes a luer connector 190 supported on the dispensing tip 128 as an alternative to the needle 140 shown in FIGS. 1-4b. The luer connector 190 may be used to form a fluid-tight connection of the first flow passage 129 with an intravenous line or other object via the luer connector 190, as will be appreciated by those skilled in the art. Notably, in this embodiment, or in a similar embodiment in which the luer connector 190 is omitted, the syringe device 100 may be used to dispense fluids directly onto tissue or other substrates as a topical applicator, if desired.

FIG. 5 further illustrates use of a different form of cap 170, namely, a cap 170 formed as a solid or other plug, e.g., of a porous material, to effectively function as a hydrophobic stopper to seal the storage chamber 132 from fluid release while simultaneously serving as a vent to allow air to enter the storage chamber when the plunger is pulled, or an elastomeric material. When used with an ampoule, the cap 170 may simply be removed from the opening 134 (if necessary), the ampoule 10 may be inserted into the storage chamber 132, and then the cap may be placed/replaced in the opening 134 to seal the storage chamber 132.

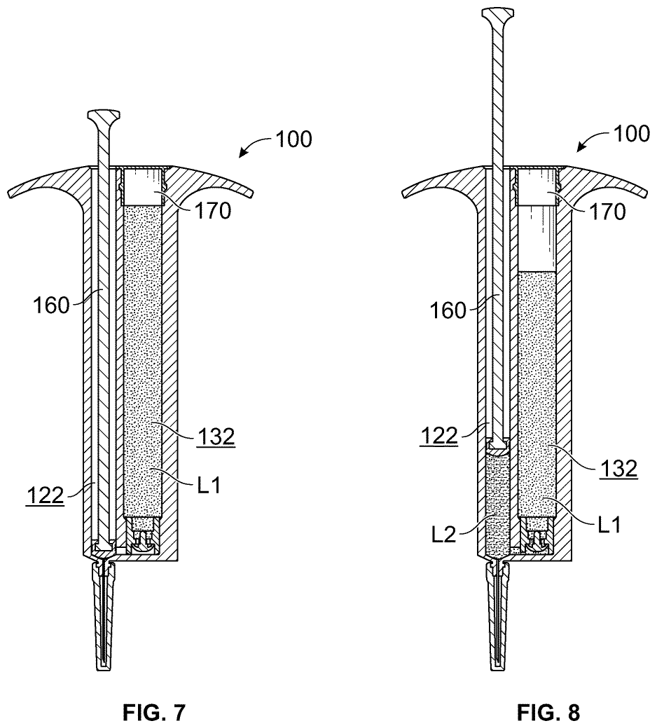

Somewhat similarly, when used with a liquid that is not packaged in an ampoule, the cap may be removed (if necessary), the liquid may be placed in the storage chamber 132, and then the cap 170 may be placed/replaced in the opening 134 to seal the storage chamber 132. FIG. 7 is a sectional view of another alternative embodiment of a syringe device 100, showing a first liquid L1 loaded directly into a storage chamber 132 of the syringe device, without an ampoule 10, and a plug-style cap 170. Accordingly, an ampoule breaker bar is omitted. This syringe device may be used to draw L1 from the storage chamber 132 into the dispensing chamber 122 by operation of plunger 160 so that L1 may be dispensed from the syringe device 100. The configuration shown in FIG. 7 may provide advantages in packaging and shipping, since the plunger 160 is fully seated within the barrel body 120 of the syringe device 100, resulting in a compact shipping package. Furthermore, this configuration may reduce the risk of inadvertent dispensing during shipping, which may occur when a plunger rod is extended and aligned directly with a prefilled fluid, in which case vibration or shock resulting from shipping may cause the plunger rod to prematurely dispense the fluid.

FIG. 8 is a sectional view of a syringe device 100 similar to that of FIG. 7, but alternatively showing a second liquid L2 loaded into the dispensing chamber 122 of the syringe device 100 for mixing with a first liquid L1 stored in the storage chamber 132 of the syringe device 100. This syringe device may be used to draw L1 from the storage chamber 132 into the dispensing chamber 122 for mixing with L2 in the dispensing chamber, by operation of plunger 160, so that a combination of L1 and L2 may be dispensed from the syringe device. Furthermore, this combination of L1 and L2 may cause the liquids to react chemically to result in desired properties for the combined fluid being dispensed. Notably, the syringe device may be pre-filled with L1 and L2, e.g., during manufacture, and be shipped in this manner for later mixing by an end user at the time of dispensing, such that the syringe device serves as the primary packaging for L1 and L2 during shipping.

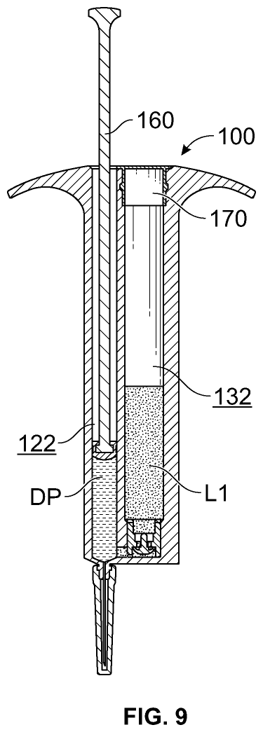

By way of further illustration, FIG. 9 is a sectional view of another syringe device 100 similar to that of FIG. 7, but alternatively showing a dry powder DP loaded into a dispensing chamber 122 of the syringe device 100 for mixing with a first liquid L1 stored in the storage chamber 132 of the syringe device. This syringe device may be used to draw L1 from the storage chamber 132 into the dispensing chamber 122 for mixing with DP in the dispensing chamber, by operation of plunger 160, so that a combination of DP and L1 (e.g., a reconstituted drug) may be dispensed from the syringe device. Notably, the syringe device may be pre-filed with DP and L1, e.g., during manufacture, and be shipped in this manner for later mixing by an end user at the time of dispensing, such that the syringe device serves as the primary packaging for the L1 and DP during shipping.

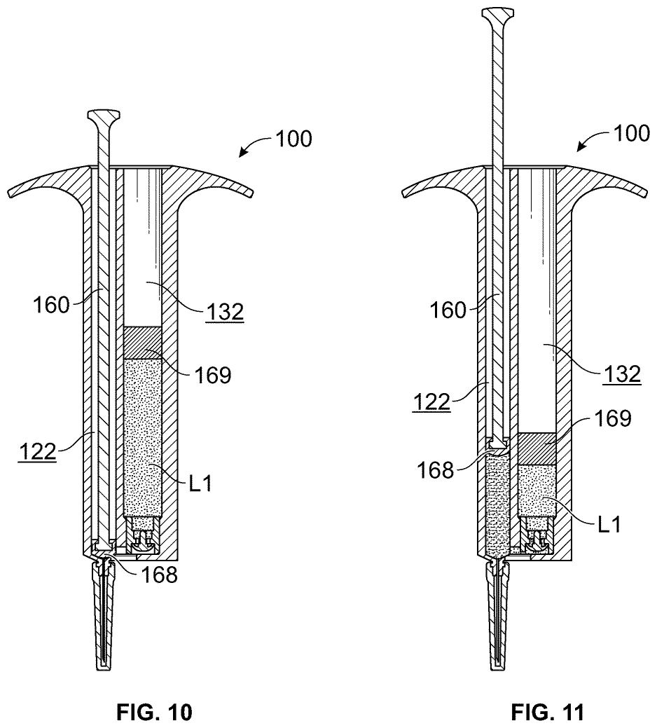

FIG. 10 is a side cross-sectional view of another alternative embodiment of a syringe device 100, showing a first liquid L1 loaded into a storage chamber 132 of the syringe device 100, and a floating plunger tip 169 in the storage chamber 132. The floating plunger tip 169 is an alternative to a plunger and may be similar in structure to plunger tip 168, but need not be configured to mate with a plunger rod. The floating plunger tip 169, in this instance, is caused to move within the storage chamber 132 by operation of the plunger 160 in the dispensing chamber 122.

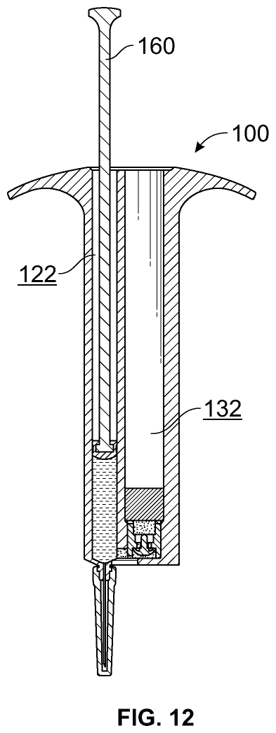

FIGS. 11 and 12 are side cross-sectional views of the syringe device of FIG. 10, showing the first liquid L1 from the storage chamber 132 being drawn into the dispensing chamber 122 of the syringe device 100. The dispensing chamber may be shipped empty (as in FIG. 10), and L1 may be drawn into the dispending chamber for dispensing via the syringe device 100, as will be appreciated from the sequence and floating plunger tip positions in FIGS. 10, 11 and 12. Alternatively, as described above with respect to FIGS. 7-9, the dispensing chamber 122 may be pre-filled with another liquid or a dry powder, such that the liquid L1 from the storage chamber 132 may be drawn into the dispensing chamber 132 for mixing with the liquid L2 or dry powder, as desired in various embodiments. The floating plunger tip 169 allows the user to transfer liquid from the storage chamber 132 into the dispensing chamber 122, irrespective of device orientation, since the plunger displaces under vacuum pressure generated by the plunger 160 without introducing air into the storage chamber 132.

As referred to above with reference to FIGS. 13 and 14, in certain embodiments, the storage body 130 and storage chamber 132 are dimensioned to receive at least two frangible ampoules. FIGS. 13 and 14 are side and top views of an alternative syringe device 100 including a storage chamber 132 configured to hold multiple ampoules. In certain embodiments at least one of the at least two frangible ampoules contains a liquid. In certain embodiments, at least one of the at least two frangible ampoules contains a dry powder. In certain embodiments, one of the frangible ampoules contains a liquid and another contains a dry powder. In certain embodiments, each of the frangible ampoules contains a respective liquid.

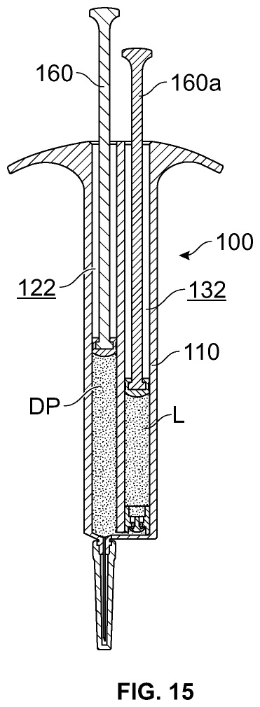

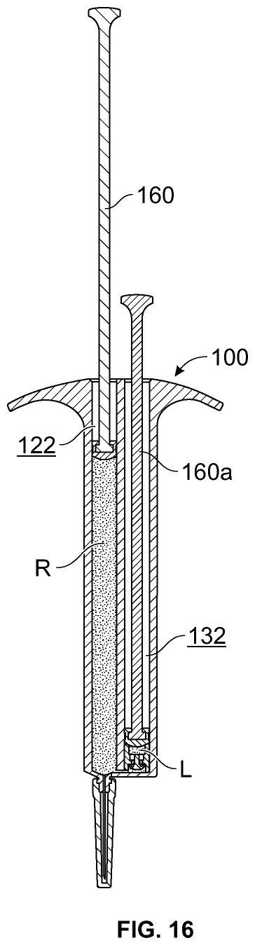

FIG. 15 is a side cross-sectional view of another alternative embodiment of a syringe device, showing a liquid L loaded into a storage chamber 132 of the syringe device 100, and a dry powder DP loaded into a dispensing chamber 122 of the syringe device 100. Alternatively, a second liquid may be contained instead of the dry powder. In this embodiment, the syringe device further includes a second plunger 160a in the storage chamber 132, in addition to the plunger 160 in the dispensing chamber 122. Accordingly, for example, the second plunger 160a may be depressed to drive the liquid L from the storage chamber 132 into the dispensing chamber 122 (where it is mixed with the dry powder DP). In response to this action, the first plunger 160 may move upwardly, as shown in FIG. 16. This causes the liquid L and dry powder DP to be mixed in the dispensing chamber 122, e.g., to form a reconstituted drug R. Then, the first plunger 160 may be depressed to dispense a reconstituted drug R (or other combined product in other embodiments, depending upon the contents of the chambers) from the syringe device 100.

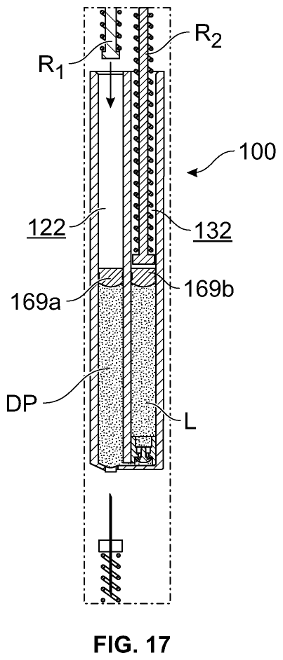

FIG. 17 is a side cross-sectional view of another alternative embodiment of a syringe device 100, which may be suitable for use, for example, with an autoinjector device, which are generally known in the art. In connection with the example of FIG. 17, the autoinjector includes multiple plunger rods R1, R2 operable to depress the first and second floating plungers 169a, 169b. In this example, a liquid L is loaded into a storage chamber 132 of the syringe device 100, and a dry powder DP is loaded into a dispensing chamber 122 of the syringe device 100. This syringe device includes a respective floating plunger tip 169a, 169b in each of the dispensing and storage chambers 122, 132. In this example, the autoinjector device is configured to actuate plunger rod R2 to cause the second floating plunger tip 169b to be depressed to drive the liquid L from the storage chamber 132 into the dispensing chamber 122 (where it is mixed with the dry powder DP). This actuation may be caused by a spring or a manual input by the user. In response to this action, the first floating plunger tip 169a may move upwardly. This causes the liquid L and dry powder DP to be mixed in the dispensing chamber 122, e.g., to form a reconstituted drug R. Then, the autoinjector is configured to actuate plunger rod R1 to cause the first floating plunger 169a to be depressed to dispense a reconstituted drug R (or other combined product in other embodiments, depending upon the contents of the chambers) from the syringe device 100.

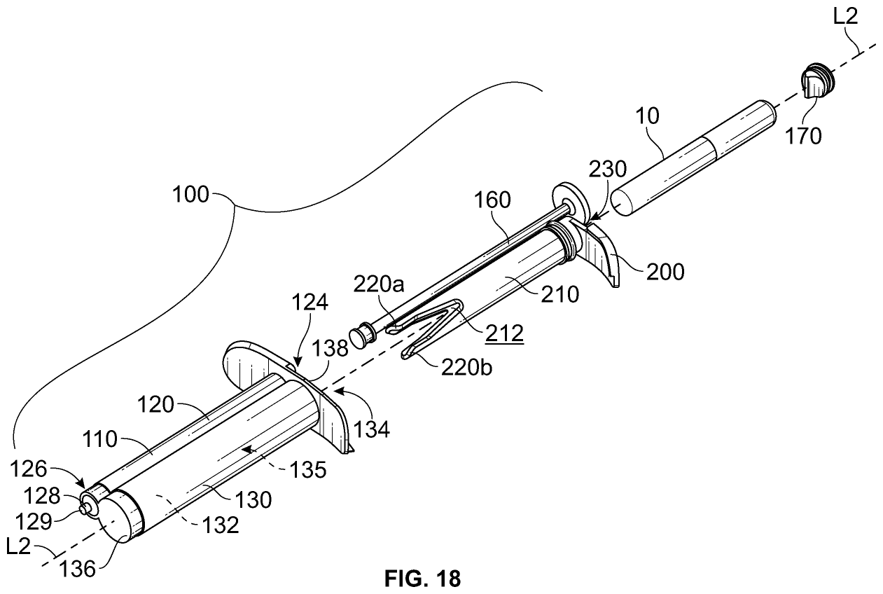

An alternative syringe device 100 in accordance with another alternative exemplary embodiment of the present invention is discussed below with reference to FIGS. 18-24. In this embodiment, an alternative to the breaker lever for rupturing frangible ampoules is provided.

Referring now to FIGS. 18-21, it will be appreciated that this exemplary syringe device 100 is similar to those shown in Figs, 1-11, but this syringe device 100 omits the pivotable breaker lever and instead includes an operable ampoule carrier 200 that includes resilient fingers 220a, 220b that are displaceable inwardly to crush a frangible ampoule contained within the ampoule carrier 200, as discussed in greater detail below.

Accordingly, this exemplary syringe device 100 includes a syringe body 110 comprising a barrel body 120 defining a dispensing chamber 122 open at a first proximal end 124 and having a first distal end 126 terminating in a dispensing tip 128 defining a first flow passage 129, similar to the embodiment of FIGS. 1-11. Further, the syringe body 110 further includes a storage body 130 defining a storage chamber 132 having a second proximal end 134 and a second distal end 136. Further, the storage body 130 defines an opening 138 at one of the second proximal end and the second distal end, and in the example of FIG. 18, at the second proximal end 134, similar to the embodiment of FIGS. 1-11. Also similar to the embodiment of FIGS. 1-11, a common wall 150 of the syringe body 110 extends from the first and second proximal ends 124, 134 to the first and second distal ends 126, 136 to separate and at least partially define both the dispensing chamber and the storage chamber, and a second flow passage is defined by the syringe body to provide fluid communication between the storage chamber and the dispensing chamber, as described above with respect to FIGS. 1-11.

Further, this exemplary embodiment includes a flow check valve supported on the syringe body and positioned in the second flow passage to resist backflow of liquid/fluid into the storage chamber, a plunger 160 positioned within the barrel body 130 for longitudinal sliding movement therein, as described in greater detail above with reference to FIGS. 1-11.

Unlike the embodiment of FIGS. 1-11, this exemplary syringe device 100 further includes an ampoule carrier body 200 having a sidewall 210 defining an ampoule chamber 212 dimensioned to receive a frangible ampoule 10. The ampoule carrier body 200, and particularly its sidewall 210, is complementary in shape to the storage chamber 132 to be rotatable therein about its longitudinal axis L2, for reasons described below.

Further, the ampoule carrier body 100, and particularly its sidewall 210, defines at least one resilient finger 220a, 220b supported in cantilevered fashion on the ampoule carrier body 200 so as to be deflectable inwardly into the ampoule chamber 212 to impinge upon and crush and ampoule 10 contained therein.

In certain embodiments, the ampoule carrier body 200 defines an ampoule opening 230, and the syringe device 100 further includes a cap 170 disposed within the ampoule opening to at least partially seal the storage opening. In certain such embodiments, the cap 170 may comprise an entry port configured to open to admit passage of a frangible ampoule into the storage chamber through the entry port, and to close to seal the storage chamber when an ampoule is not positioned within the entry port, and the entry port may be constructed of an elastomeric material and configured as a duckbill valve, in a manner similar to that described above.

In accordance with the present invention, in this embodiment, the resilient finger(s) 220a, 220b and an interior wall 135 of the storage body 130 are configured complimentarily such that rotation of the ampoule carrier body 200 within the storage chamber, about its longitudinal axis L2, causes the resilient finger(s) 220a, 220b to be displaced inwardly into the ampoule chamber 212 to selectively crush any frangible ampoule 10 contained therein.

FIG. 22 is a cross-sectional view of the syringe device 100 taken along line 22-22 of FIG. 21. FIG. 23 is an enlarged cross-sectional view of a portion of FIG. 22. FIG. 27 is a cross-sectional view taken along line 27-27 of FIG. 26. FIG. 28 is an enlarged cross-sectional view of portion of FIG. 27. Accordingly, FIGS. 22 and 23 show arrangement of the resilient fingers 220a, 220b within the storage body 130.

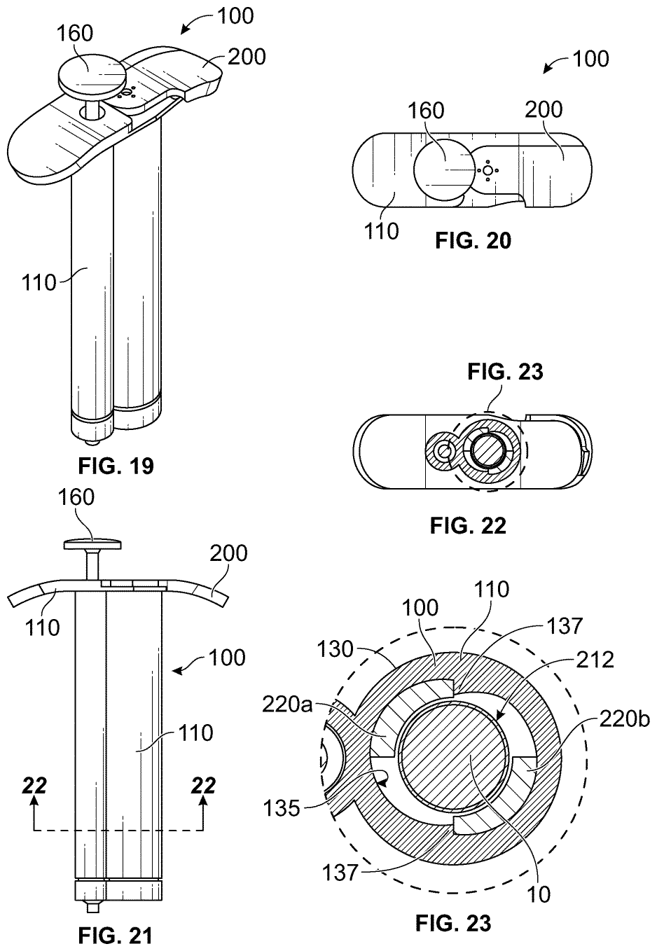

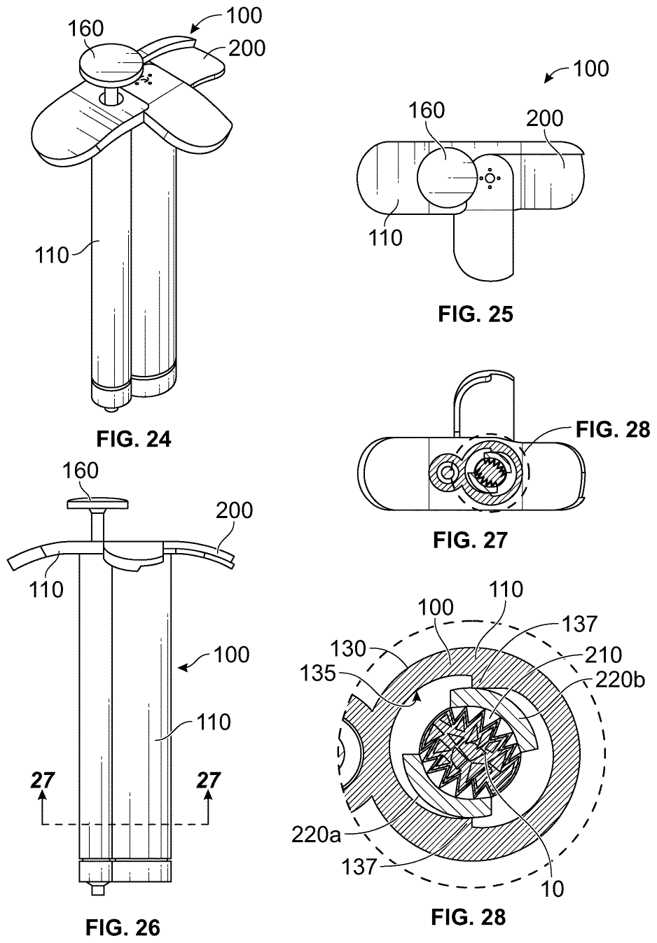

FIGS. 19-23 show the syringe device 100 in a stored/storage state, and FIGS. 24-28 shown the syringe device 100 is an ampoule break state. As will be appreciated from FIGS. 22 and 23, the interior wall 135 of the storage body 130 defines at least a portion of the storage chamber 132 that is non-circular in transverse cross-section. In the stored/storage state, the resilient finger(s) 220a, 220b do not interfere with the non-circular cross-section, and they are not displaced inwardly into the ampoule chamber 212. The non-circular portion causes the resilient finger(s) 220a, 220b to interfere with the non-circular portion and be displaced inwardly into the ampoule chamber 212 when the ampoule carrier body 200 is rotated within the storage chamber 130. In this exemplary embodiment, the interior wall 135 of the storage body 130 defines ribs 137 that serve as ramps that displace the fingers 220a, 220b inwardly as the carrier body 200 is rotated about its longitudinal axis to cause the fingers 220a, 220b to ride up the ramps and be displaced inwardly by the increasing height of the ramps as the carrier body is rotated.

In certain embodiments, the ampoule carrier body comprises at least one rib extending radially away from a longitudinal axis of the ampoule carrier body. In such a case, at least one rib of the ampoule carrier body does not interfere with the interior wall of the storage chamber in a first angular position of the ampoule carrier body within the storage chamber, and does interfere with the interior wall of the storage chamber in a second angular position of the ampoule carrier body within the storage chamber to cause deflection of the at least one resilient finger sufficient to crush the frangible ampoule contained in the ampoule chamber.

In certain embodiments, the orientation of the top flange of the carrier body and the arrangement of the at least one flexible finger can be reversed. In other words, the fingers may be relaxed when the flange is pivoted outward/misaligned (as in FIGS. 24 and 25) and the fingers may be in the deflected/flexed state to crush the ampoule when the tab is in the fully seated/aligned position (as in FIGS. 19 and 20).

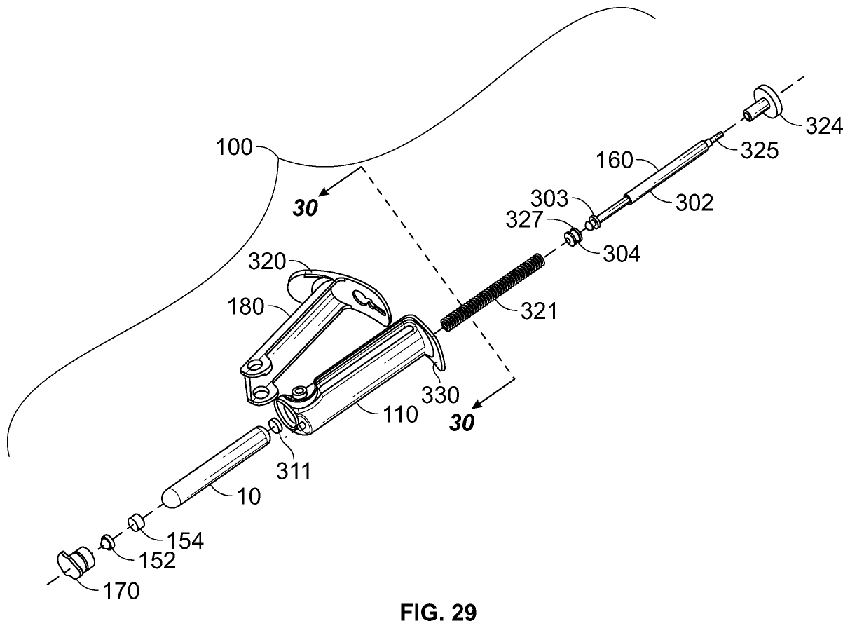

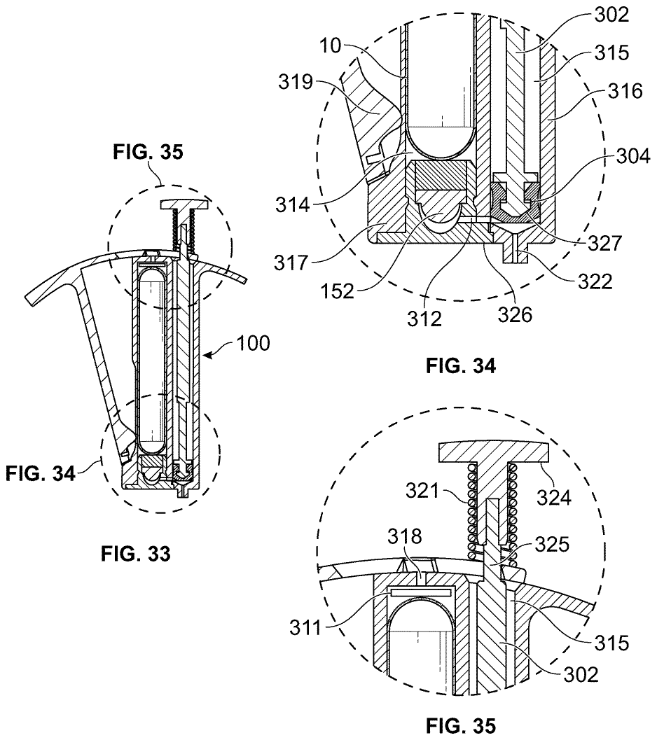

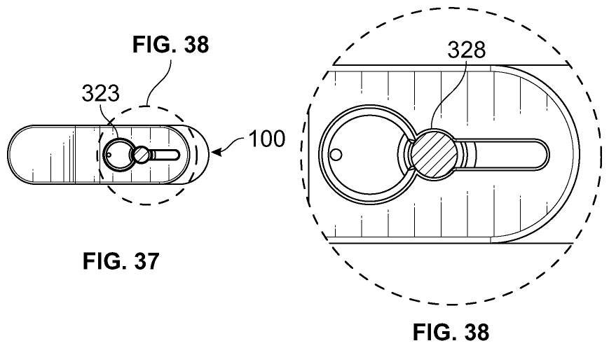

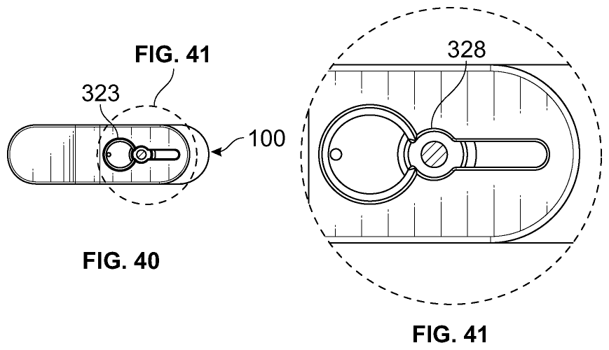

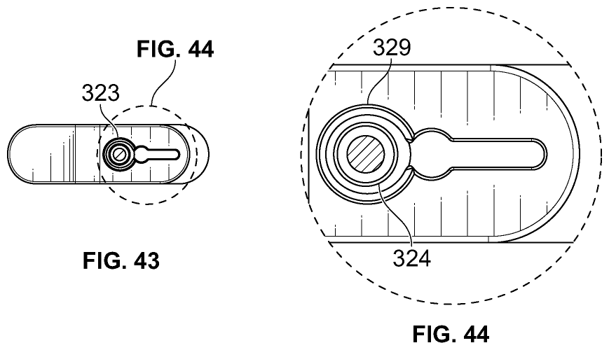

Another alternative syringe device 100 in accordance with another alternative exemplary embodiment of the present invention is discussed below with reference to FIGS. 29-44. In this embodiment, a breaker lever for rupturing frangible ampoules is provided in a manner similar to that described above with reference to FIGS. 1-11, and in addition, a spring-biased mechanism is provided for automatedly moving the plunger to draw a vacuum and draw liquid from a fractured ampoule in the storage chamber 132 into the dispensing chamber 122. Referring now to FIG. 29, an exploded view of this embodiment of the syringe device 100 is shown. This syringe device 100 is adapted to house a conventional, commercially-available crushable ampoule 10. Further, the ampoule syringe device 100 is comprised of several components: a piston head 324, piston 302, plunger tip 304, priming spring 321, syringe body 110, breaker lever 180, vent in the nature of a hydrophobic filter 311, ampoule 10, particulate filter 154, check valve 152 and cap 170.

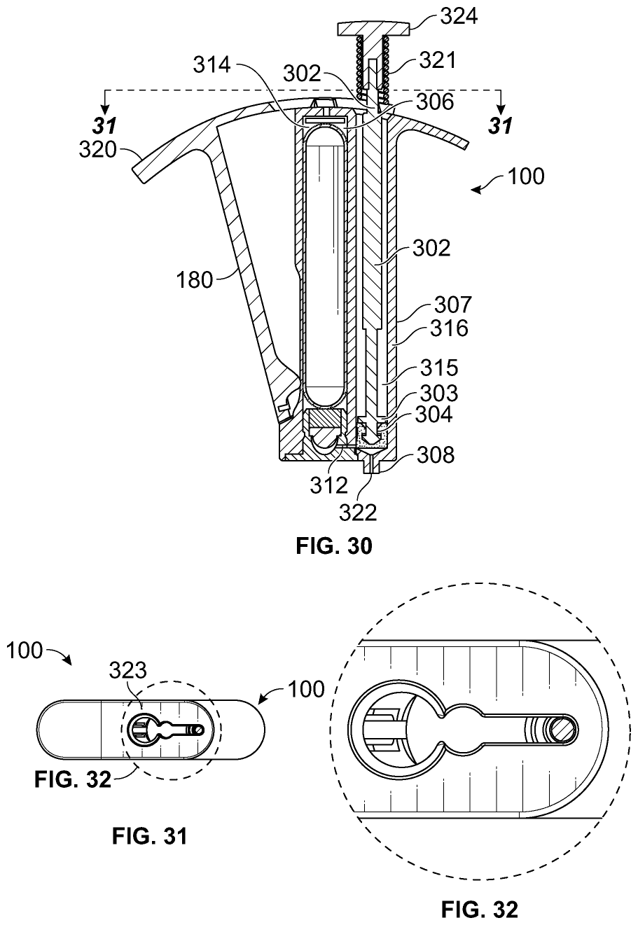

This syringe device 100 is shown in a storage state, and the ampoule 10 in an intact state, in FIGS. 30-35. As will be appreciated from FIG. 29, the exemplary ampoule syringe device 100 has a construction somewhat similar to a conventional syringe, in that it includes a plunger 160 carrying a plunger tip 168 that rides within and forms a fluid-tight seal with an interior wall 316 of a barrel body 307 to define a dispensing chamber 315, which is somewhat analogous to the portion of a syringe that conventionally contains drugs/fluids. Similar to some conventional syringes, the barrel body 307 terminates in a dispensing tip 308 defining a fluid flow passage 322 in fluid communication with the dispensing chamber 315. In some embodiments, the tip is configured with a Luer-lock collar to enable connection of a needle and facilitate the delivery of intramuscular, intravenous, and/or subcutaneous modes. In other embodiments, a needle may be staked or otherwise supported into fluid flow passage 322 to facilitate fluid communication with dispensing chamber 315.

Referring now to FIGS. 30, 31 and 32, in contrast to most conventional syringes, barrel body 307 is adjoined by storage body/ampoule enclosure 306, which defines an internal ampoule chamber 314. For reasons discussed herein, ampoule storage body/ampoule enclosure 306 may be constructed from a plastic material wherein the majority of the main body is rigid, and the lower portion of the body is thinner and compressible/displaceable. Further, the ampoule chamber 314 is in fluid communication with the dispensing chamber 315 via second flow passage 312. Further, in a storage state, the exemplary ampoule syringe device 100 comprises a pivotably-mounted breaker lever 180, which is connected to a remainder of the exemplary syringe device 100 by hinge 317 (which may be a living hinge of continuous flexible material), offset from the main body by an acute angle, and contains breaker protrusion 319, breaker flange 320 and breaker flange channel 323.

Further, in accordance with the present invention, the barrel body 307, forms the dispensing chamber 315, which is enclosed by the interior wall 316. This dispensing chamber 315 is dimensioned to accommodate the plunger rod/piston 302 consisting of a plunger tip 304, a piston flange 303, piston head 324 and piston neck 325. The piston 302 includes a priming spring 321, which is concentrically pre-assembled onto the piston 302 and operable to drive the plunger to draw liquid from the ampoule into the dispensing chamber 315, as described in greater detail below. Prior to assembly, the breaker lever 180 may be positioned such that it is adducted against the main body of storage body/ampoule enclosure 306. This positioning facilitates the alignment of the breaker flange channel 323 and the dispensing chamber 315, allowing the piston 302 to be inserted through both passages. During the assembly process, the piston 302 and priming spring 321 assembly is inserted through the breaker flange channel 323, with the priming spring 321 positioned near the piston head 324. The priming spring 321 is held in a compressed state against the piston head 324 while the piston is inserted through the breaker flange channel 323 and into the dispensing chamber 315. Once the piston 302 is inserted, the breaker lever 180 is abducted from the ampoule enclosure 306 until the breaker flange channel 323 engages the piston neck 325, causing mechanical interference that locks the piston 302 in place and prevents the priming spring 321 from expanding.

Further, in accordance with the present invention, the ampoule enclosure 306 is designed to receive components from the bottom, e.g., in the following order: the hydrophobic filter 311, the ampoule 10, the particle filter 154, and the check valve 152. This sequence indicates an exemplary order of assembly. The ampoule cap 170 secures the components within the ampoule enclosure 306 and can be attached using ultrasonic welding, adhesive, or a mechanical snap fit. The top of the ampoule enclosure 306 features a vent opening 318, which permits air to enter the ampoule chamber 314 while the hydrophobic filter 311 prevents fluid from the ampoule 10 from leaking out.

In accordance with the present invention, the plunger tip 304 may be formed of a resilient/elastomeric material. The plunger tip 304 includes at least one sealing rib 327 that is shaped to maintain an annular seal between the piston 302 and the interior wall 316 of the dispensing chamber 315 of the cylinder body 307. Accordingly, the plunger tip 304 and sealing rib(s) 327 are shaped to correspond to a shape of the interior wall 316 of the cylinder body 307. In the storage state, the plunger tip 304 rests at the bottom of the dispensing chamber 315, preventing fluid communication between the first fluid flow passage 322 and the second fluid flow passage 312. Further, the distal end of the plunger tip 304 is shaped to correspond to the rounded bottom portion of the dispensing chamber 315.

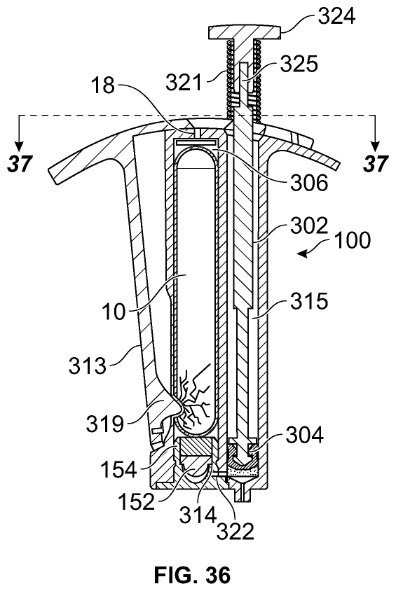

The syringe device 100 is shown in a cracking state, and the ampoule 10 in a ruptured state, in FIGS. 36-38. To achieve this cracking state, the breaker lever 180 is adducted toward ampoule enclosure 306 such that breaker flange channel 323 travels along piston neck 325 until release notch 328 aligns axially with piston 302 and piston head 324. Simultaneously, the breaker protrusion 319 induces deflection of the ampoule enclosure 306 due to the material at the bottom of the enclosure being thinner than at the top. This resulting compression causes the ampoule 10 to fracture. Concurrently, the breaker flange channel 323 is disposed to disengage from the piston neck 325, alleviating the mechanical interference that locks the piston 302 in place and prevents the priming spring 321 from expanding, and thus causes the priming spring 321 to expand. As the priming spring 321 expands, it exerts an upward force against the piston head 324, causing the piston 302 and plunger tip 304 to move upwardly with respect to the dispensing chamber 315, creating vacuum pressure to draw and facilitate the flow of liquid exiting the cracked ampoule 10 from the ampoule chamber 314 through the particle filter 154, which captures glass fragments, thereby preventing them from exiting ampoule chamber 314. Concurrently, ambient air is drawn into the ampoule chamber 314 via the vent opening 318 in ampoule enclosure 306. Subsequently, the liquid flows through a check valve 152 (e.g., a duckbill-style valve, or alternatively, an umbrella-style valve or poppett valve), which permits one-way flow of the liquid from the ampoule chamber 314 through the second flow passage 312 and into the dispensing chamber 315.

In an alternative embodiment, the ampoule may contain contents that are part liquid formulation and part compressed gas. In such an alternative embodiment, the vent/air valve 318 may be omitted. Upon rupturing the ampoule, the compressed gas escapes and forces the liquid from the ampoule chamber into the syringe chamber. The compressed gas generates a positive pressure that causes the piston to extend in a similar manner, to advance the liquid/fluid through the first flow passage, as previously discussed without requiring the compression spring.

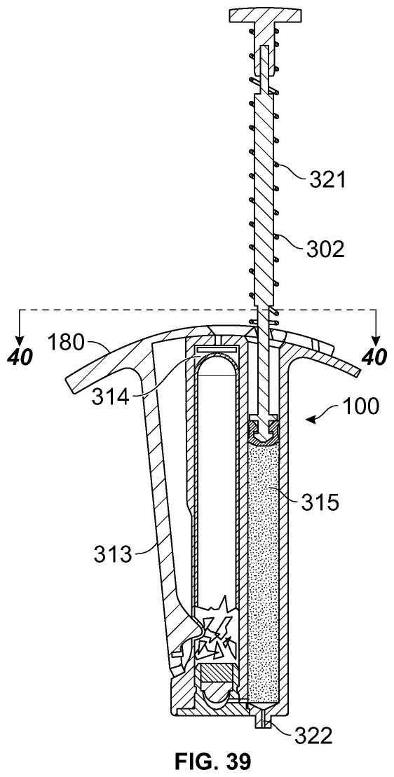

The syringe device 100 is shown in a vacuum state, with the ampoule 10 in a ruptured condition, in FIGS. 39-41. In this state, the piston 302 is fully retracted due to the expansion of the priming spring 321, e.g., to its natural length. Consequently, the liquid has been evacuated from the ampoule chamber 314 and suctioned into the dispensing chamber 315. In this state, the priming spring 321 rests atop the breaker flange channel 323, as its outer diameter is greater than the inner diameter of the release notch 328. In some embodiments, a sealed Luer-lock collar (not shown) may be pre-assembled on the dispensing tip 308 to prevent air from entering the dispensing tip 308 via the first fluid flow passage 322.

It should be noted that in other embodiments, the tip may be effectively sealed other than by a Luer-lock collar, and yet the same result may be achieved. For example, a pre-staked needle may be molded into the dispensing tip 308 wherein the needle may be equipped with a removable, airtight sterile barrier.

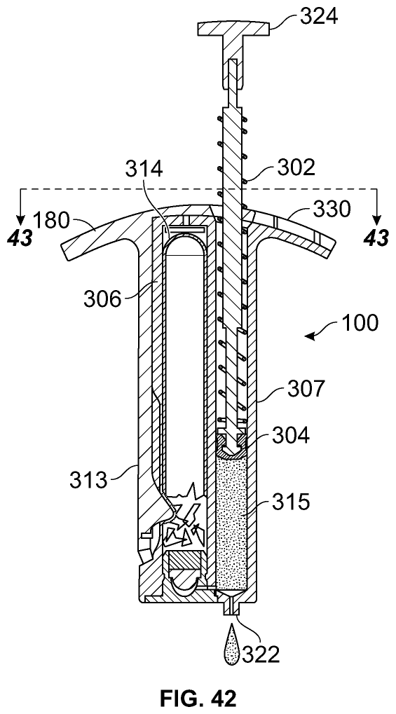

After the liquid has been transferred from the ampoule chamber 314 of into the dispensing chamber 315 of the cylinder body 307, the user adducts breaker lever 180 toward ampoule enclosure 306 causing the breaker flange channel 323 to travel along piston 302 until dispense notch 329 aligns axially with piston 302, piston head 324 and dispensing chamber 315, as will be appreciated by FIGS. 42-44. In this position, the dimension of the dispense notch 329 exceeds the dimension of the priming spring 302, which causes the priming spring 302 to extend within the dispensing chamber 315. With the priming spring 302 in this extended position, the user has control over the dispensing process without needing to overcome the resistance of additional spring force. The liquid/fluid may then be dispensed with precision by manually grasping the syringe device 100 (e.g., after purging the chamber of air using a conventional technique), which may involve grasping of the breaker flange 320 and the syringe flange 330 with a single hand such that the flanges rest between a user's forefinger and middle finger with the thumb positioned atop the piston head 324. Dispensing the liquid requires a downward axial force applied by the user's thumb such that the plunger tip 304 approaches the bottom of the dispense chamber as indicated in FIGS. 42-44.

Notably, this type of syringe operation action, and manual advancing of the plunger relative to the cylinder body, is a familiar type of manual task, and is well-suited to precise manual control, and therefore to precise dispensing of fluid from the device in an intended manner, to help ensure accurate/intended fluid volume delivery.

Further, precise dose delivery is facilitated by the incorporation of dose markings that adhere to established industry standards. The ergonomic configuration of the breaker flange 320 and syringe flange 330 further enhance user comfort and accuracy during administration.

The syringe device 100 in the dispensed state results in full axial translation of the plunger tip 304 such that the plunger tip 304 reaches the bottom of the dispensing chamber 315, dispensing the liquid via the fluid flow passage 322. The breaker lever 180 remains fully adducted to the ampoule enclosure 306.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1-3. (canceled)

4. A syringe device for dispensing fluids, the syringe device comprising:
a syringe body comprising:
a barrel body defining a dispensing chamber open at a first proximal end and having a first distal end terminating in a dispensing tip defining a first flow passage, the dispensing chamber being in fluid communication with the first flow passage, the dispensing chamber being elongated to have a first longitudinal axis; and
a storage body defining a storage chamber having a second proximal end and a second distal end, the storage chamber defining an opening at one of the second proximal end and the second distal end, the storage chamber being elongated to have a second longitudinal axis distinct from the first longitudinal axis;
a flow check valve supported on the syringe body, the flow check valve being positioned to resist backflow of liquid into the storage chamber via the second flow passage;
a plunger positioned within the barrel body for longitudinal sliding movement therein, the plunger and extending beyond the first proximal end of the barrel body, the plunger comprising:
a plunger rod having a third proximal end and a third distal end; and
a plunger tip supported on the third distal end of the plunger rod, the plunger tip being configured to abut and seal with an internal sidewall of the barrel body; and
a cap disposed within the opening at a respective one of the second proximal end and the second distal end of the storage body to seal the storage chamber;
wherein a common wall of the syringe body extends from the first and second proximal ends to the first and second distal ends to separate and at least partially define both the dispensing chamber and the storage chamber, and wherein a second flow passage is defined by the syringe body to provide fluid communication between the storage chamber and the dispensing chamber;
wherein the cap comprises an entry port configured to open to admit passage of a frangible ampoule into the storage chamber through the entry port, and to close to seal the storage chamber when an ampoule is not positioned within the entry port; and
wherein the entry port is constructed of an elastomeric material and configured as a duckbill valve.

5-16. (canceled)

17. A syringe device for dispensing fluids, the syringe device comprising:
a syringe body comprising:
a barrel body defining a dispensing chamber open at a first proximal end and having a first distal end terminating in a dispensing tip defining a first flow passage, the dispensing chamber being in fluid communication with the first flow passage, the dispensing chamber being elongated to have a first longitudinal axis; and
a storage body defining a storage chamber having a second proximal end and a second distal end, the storage chamber defining an opening at one of the second proximal end and the second distal end, the storage chamber being elongated to have a second longitudinal axis distinct from the first longitudinal axis;
a flow check valve supported on the syringe body, the flow check valve being positioned to resist backflow of liquid into the storage chamber via the second flow passage;
a plunger positioned within the barrel body for longitudinal sliding movement therein, the plunger and extending beyond the first proximal end of the barrel body, the plunger comprising:
a plunger rod having a third proximal end and a third distal end; and
a plunger tip supported on the third distal end of the plunger rod, the plunger tip being configured to abut and seal with an internal sidewall of the barrel body; and
a cap disposed within the opening at a respective one of the second proximal end and the second distal end of the storage body to seal the storage chamber;
a first liquid disposed in the storage chamber, the first liquid being contained in a frangible ampoule positioned within the storage chamber, at least two frangible ampoules being positioned within the storage chamber, at least one of the at least two frangible ampoules contains the first liquid; and
a dry powder disposed in the dispensing chamber;
wherein a common wall of the syringe body extends from the first and second proximal ends to the first and second distal ends to separate and at least partially define both the dispensing chamber and the storage chamber, and wherein a second flow passage is defined by the syringe body to provide fluid communication between the storage chamber and the dispensing chamber; and
wherein at least one of the at least two frangible ampoules contains a dry powder.

18-22. (canceled)

23. A syringe device for dispensing fluids, the syringe device comprising:
a syringe body comprising:
a barrel body defining a dispensing chamber open at a first proximal end and having a first distal end terminating in a dispensing tip defining a first flow passage, the dispensing chamber being in fluid communication with the first flow passage, the dispensing chamber being elongated to have a first longitudinal axis; and
a storage body defining a storage chamber having a second proximal end and a second distal end, the storage chamber defining an opening at one of the second proximal end and the second distal end, the storage chamber being elongated to have a second longitudinal axis distinct from the first longitudinal axis, an outer wall of the storage body at least partially defining the storage chamber, the outer wall defining a flexible portion displaceable inwardly to apply pressure to a frangible ampoule positioned within the storage chamber;
wherein a common wall of the syringe body extends from the first and second proximal ends to the first and second distal ends to separate and at least partially define both the dispensing chamber and the storage chamber, and wherein a second flow passage is defined by the syringe body to provide fluid communication between the storage chamber and the dispensing chamber;
a flow check valve supported on the syringe body, the flow check valve being positioned in the second flow passage to resist backflow of liquid into the storage chamber;
a plunger positioned within the barrel body for longitudinal sliding movement therein, the plunger and extending beyond the first proximal end of the barrel body, the plunger comprising:
a plunger rod having a third proximal end and a third distal end; and
a plunger tip supported on the third distal end of the plunger rod, the plunger tip being configured to abut and seal with an internal sidewall of the barrel body;
a breaker lever mounted to the syringe body, the breaker lever defining a protrusion positioned and configured to apply force to the flexible portion of the outer wall to displace the outer wall inwardly and break a frangible ampoule positioned within the storage chamber when the breaker lever is adducted toward the storage chamber, the breaker lever being operable between at a stored position and an ampoule break position; and
a cap disposed within the opening at a respective one of the second proximal end and the second distal end of the storage body to seal the storage chamber.

24. The syringe device of claim 23, wherein the breaker lever is constructed of a rigid material and is pivotably interconnected to the syringe body by a pivot joint.

25. The syringe device of claim 23, wherein the breaker lever is integrally formed with the syringe body, the breaker lever being pivotably interconnected to the syringe body by a living hinge.

26. The syringe device of claim 23, wherein the cap comprises an entry port configured to open to admit passage of a frangible ampoule into the storage chamber through the entry port, and to close to seal the storage chamber when an ampoule is not positioned within the entry port.

27. The syringe device of claim 26, wherein the entry port is constructed of an elastomeric material and configured as a duckbill valve.

28. The syringe device of claim 23, further comprising a particulate filter supported on the syringe body within at least one of the first flow passage and the second flow passage.

29. The syringe device of claim 23, wherein the flexible portion of the outer wall has a thickness that is reduced relative to a respective thickness of an adjacent portion the outer wall.

30. The syringe device of claim 23, wherein the storage chamber is dimensioned to receive at least two frangible ampoules.

31. The syringe device of claim 23, wherein the breaker lever is operable in at a stored position, an ampoule break and vacuum position, and a dispense position;
wherein the breaker lever defines a top flange having a channel defining a plurality of spaced slots, each of the plurality of slots being dimensioned and positioned to facilitate storage, ampoule break and vacuum, and dispense functions corresponding to each of the stored, ampoule break and vacuum, and dispense positions.

32. The syringe device of claim 31, further comprising:
a spring biasing the plunger to cause the plunger to automatically draw a vacuum when the breaker lever is in the ampoule break and vacuum position.

33. The syringe device of claim 32, wherein the plunger defines a neck, wherein interaction between the neck, the plurality of slots and the top flange selectively secures the plunger, thereby preventing its longitudinal movement within the barrel body until ready for use;
whereby in the stored position, the frangible ampoule remains intact and no vacuum may be drawn; and
whereby in the ampoule break and vacuum position, the frangible ampoule is ruptured and the spring is extended to draw a vacuum in the dispensing chamber.

34. The syringe device of claim 33, wherein an outer diameter of the spring exceeds a diameter of a breaker slot in the ampoule break and vacuum position.

35. The syringe device of claim 32, wherein in the dispense position, curvature of the breaker's top flange provides a consistent and ergonomic grip for a user.

36. The syringe device of claim 32, wherein the frangible ampoule stores a liquid and a compressed gas that automatically generates a positive pressure operable to advance the liquid through the first flow passage according to a respective position of the breaker lever.

37. A syringe device for dispensing fluids, the syringe device comprising:
a syringe body comprising:
a barrel body defining a dispensing chamber open at a first proximal end and having a first distal end terminating in a dispensing tip defining a first flow passage, the dispensing chamber being in fluid communication with the first flow passage, the dispensing chamber being elongated to have a first longitudinal axis; and
a storage body defining a storage chamber having a second proximal end and a second distal end, the storage body defining an opening at one of the second proximal end and the second distal end, the storage chamber being elongated to have a second longitudinal axis distinct from the first longitudinal axis;
wherein a common wall of the syringe body extends from the first and second proximal ends to the first and second distal ends to separate and at least partially define both the dispensing chamber and the storage chamber, and wherein a second flow passage is defined by the syringe body to provide fluid communication between the storage chamber and the dispensing chamber;
a flow check valve supported on the syringe body, the flow check valve being positioned in the second flow passage to resist backflow of liquid into the storage chamber;
a plunger positioned within the barrel body for longitudinal sliding movement therein, the plunger and extending beyond the first proximal end of the barrel body, the plunger comprising:
a plunger rod having a third proximal end and a third distal end; and
a plunger tip supported on the third distal end of the plunger rod, the plunger tip being configured to abut and seal with an internal sidewall of the barrel body; and
an ampoule carrier body defining an ampoule chamber dimensioned to receive a frangible ampoule, the ampoule carrier body being complementary in shape to the storage chamber to be rotatable therein about its longitudinal axis, the ampoule carrier body defining at least one resilient finger, the at least one resilient finger and an interior wall of the storage body being configured complimentarily such that rotation of the ampoule carrier body within the storage chamber, about its longitudinal axis, causes the least one resilient finger to be displaced inwardly into the ampoule chamber to selectively crush the frangible ampoule contained therein.

38. The syringe device of claim 37, wherein the interior wall defining at least a portion of the storage chamber is non-circular in transverse cross-section, the non-circular portion causing the at least one resilient finger to be displaced inwardly into the ampoule chamber when the ampoule carrier body is rotated within the storage chamber.

39. The syringe device of claim 37, wherein the ampoule carrier body comprises at least one rib extending radially away from a longitudinal axis of the ampoule carrier body, wherein the at least one rib of the ampoule carrier body does not interfere with the interior wall of the storage chamber in a first angular position of the ampoule carrier body within the storage chamber, and wherein the at least one rib of the ampoule carrier body does interfere with the interior wall of the storage chamber in a second angular position of the ampoule carrier body within the storage chamber to cause deflection of the at least one resilient finger sufficient to crush the frangible ampoule contained in the ampoule chamber.

40. The syringe device of claim 37, wherein the carrier body defines an ampoule opening, the syringe device further comprising:
a cap disposed within the ampoule opening to at least partially seal the storage opening.

41. The syringe device of claim 40, wherein the cap comprises an entry port configured to open to admit passage of a frangible ampoule into the storage chamber through the entry port, and to close to seal the storage chamber when an ampoule is not positioned within the entry port.

42. The syringe device of claim 41, wherein the entry port is constructed of an elastomeric material and configured as a duckbill valve.

43. The syringe device of claim 37, further comprising a particulate filter supported on the syringe body within at least one of the first flow passage and the second flow passage.

44. The syringe device of claim 37, wherein the storage chamber is dimensioned to receive at least two frangible ampoules.
